(19) United States
(12) Patent Application Publication
(10) Pub. No.: US 2022/0125839 A1
(43) Pub. Date: Apr. 28, 2022

(54) COMBINATION OF T-CELL THERAPY AND TARGETED THERAPY FOR TREATING THERAPY-RESISTANT MELANOMA WITH MUTATIONS IN THE BRAF GENE

(71) Applicant: H. LEE MOFFITT CANCER CENTER AND RESEARCH INSTITUTE, INC., Tampa, FL (US)

(72) Inventors: Amod SARNAIK, Tampa, FL (US); Dmitri I. GABRILOVICH, Villanova, PA (US)

(21) Appl. No.: 17/430,331

(22) PCT Filed: Feb. 13, 2020
(86) PCT No.: PCT/US2020/018065

§ 371 Date: Aug. 12, 2021

Related U.S. Application Data
(60) Provisional application No. 62/805,220, filed on Feb. 13, 2019.

(51) Int. Cl. A61K 35/17; A61P 35/00; A61K 31/44; A61K 31/506; A61K 31/137

(52) U.S. Cl. CPC A61K 35/17; A61P 35/00; A61K 31/137; A61K 31/506; A61K 31/44

(57) ABSTRACT

Approximately 50% of melanoma patients carry a mutation in the BRAF protein. Targeted therapy with inhibitors of BRAF and the downstream pathway is very effective in these patients, but long-term benefits are limited due to the onset of therapy resistance. Previous studies demonstrated that BRAF inhibitors (BRAFi) positively affect the antitumor immune response mediated by T cells. Disclosed are methods of treating, preventing, inhibiting, reducing, and/or ameliorating a cancer and/or metastasis in a subject using an adoptive T cell therapy, the method comprising administering to the subject a BRAF inhibitor (BRAFi) (such as, for example, sorafenib, vemurafenib, dabrafenib, and/or encorafenib) and an adoptive T cell therapy, wherein administration of the BRAF inhibitor increases insulin-like growth factor II receptor (IGF2R).

This application claims the benefit of U.S. Provisional Application No. 62/805,220, filed on Feb. 13, 2019, which is incorporated herein by reference in its entirety. This invention was made with government support under Grant Nos. 5P01CA114046, 1U54CA224070, P50CA168536, and P50CA174523 awarded by National Institutes of Health. The government has certain rights in the invention.

### I. BACKGROUND

Approximately 50% of melanoma patients carry a mutation in the BRAF protein. Targeted therapy with inhibitors of BRAF and the downstream pathway is very effective in these patients, but long-term benefits are limited due to the onset of therapy resistance. Previous studies demonstrated that BRAF inhibitors (BRAFi) positively affect the antitumor immune response mediated by T cells. Accordingly, what are needed are advanced combination therapeutic approaches that take advantage the of the efficacy of BRAFi to achieve a more clinically beneficial outcome.

### II. SUMMARY

Disclosed are methods for treating cancer using BRAF and/or MEK inhibitors in combination with adaptive T cell therapy.

In one aspect, disclosed herein are methods of treating, preventing, inhibiting, reducing, and/or ameliorating a cancer and/or metastasis in a subject using an adoptive T cell therapy, the method comprising administering to the subject a BRAF inhibitor (BRAFi) (such as, for example, sorafenib, vemurafenib, dabrafenib, and/or encorafenib) and an adoptive T cell therapy, wherein administration of the BRAF inhibitor increases insulin-like growth factor II receptor (IGF2R).

Also disclosed herein are methods of increasing the sensitivity of a cancer in a subject to adoptive T cell therapy, the method comprising administering to the subject receiving adoptive T cell therapy a BRAF inhibitor (BRAFi) (such as, for example, sorafenib, vemurafenib, dabrafenib, and/or encorafenib), wherein administration of the BRAF inhibitor increases insulin-like growth factor II receptor (IGF2R).

In one aspect disclosed herein are methods of treating, preventing, inhibiting, reducing, and/or ameliorating a cancer and/or metastasis of any preceding aspect or methods of increasing the sensitivity of a cancer in a subject to adoptive T cell therapy of any preceding aspect said methods comprising administering to the subject a BRAFi, wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer, colon cancer, rectal cancer, prostatic cancer, or pancreatic cancer melanoma, colorectal carcinoma, papillary thyroid carcinoma, hairy cell leukemia, and Langerhans cell histiocytosis, pleomorphic xanthoastrocytoma, ganglioglioma, epithelioid glioblastoma, and gliomas diagnosed at a younger age; melanoma, colorectal, thyroid, and Non-small cell lung cancer (NSCLC), as well as hairy cell leukemia. In one aspect, it is understood and herein contemplated that the cancer is a BRAF inhibitor resistant cancer.

Also disclosed herein are methods of treating, preventing, inhibiting, reducing, and/or ameliorating a cancer and/or metastasis of any preceding aspect or methods of increasing the sensitivity of a cancer in a subject to adoptive T cell therapy of any preceding aspect said methods comprising administering to the subject a BRAFi, wherein the adoptively transferred T cells are chimeric antigen receptor T cells (CAR T cells) or tumor infiltrating lymphocytes (TlLs).

In one aspect, disclosed herein are methods of treating, preventing, inhibiting, reducing, and/or ameliorating a cancer and/or metastasis in a subject with an adoptive T cell therapy, the method comprising administering to the subject an lGF2R agonist (such as, for example, clenbuterol) and an adoptive T cell therapy.

Also disclosed herein are methods of increasing the sensitivity of a cancer in a subject to adoptive T cell therapy, the method comprising administering to the subject an lGF2R agonist (such as, for example, clenbuterol).

In one aspect, disclosed herein are methods of treating, preventing, inhibiting, reducing, and/or ameliorating a cancer and/or metastasis in a subject of any preceding aspect or methods of increasing the sensitivity of a cancer in a subject to adoptive T cell therapy of any preceding aspect said methods comprising administering to the subject an lGF2R agonist, wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer melanoma, colorectal carcinoma, papillary thyroid carcinoma, hairy cell leukemia, and Langerhans cell histiocytosis, pleomorphic xanthoastrocytoma, ganglioglioma, epithelioid glioblastoma, and gliomas diagnosed at a younger age; melanoma, colorectal, thyroid, and Non-small cell lung cancer (NSCLC), as well as hairy cell leukemia. In one aspect, it is understood and herein contemplated that the cancer is a BRAF inhibitor resistant cancer.

Also disclosed herein are methods of treating, preventing, inhibiting, reducing, and/or ameliorating a cancer and/or metastasis in a subject of any preceding aspect or methods of increasing the sensitivity of a cancer in a subject to adoptive T cell therapy of any preceding aspect said methods comprising administering to the subject an lGF2R agonist, wherein the adoptively transferred T cells are chimeric antigen receptor T cells (CAR T cells) or tumor infiltrating lymphocytes (TlLs).

In one aspect, disclosed herein are methods of treating, preventing, inhibiting, reducing, and/or ameliorating a cancer and/or metastasis in a subject of any preceding aspect or methods of increasing the sensitivity of a cancer in a subject to adoptive T cell therapy of any preceding aspect said methods comprising administering to the subject an lGF2R agonist, further comprising administering to the subject a BRAF inhibitor.

Also disclosed herein are methods of treating, preventing, inhibiting, reducing, and/or ameliorating a cancer and/or metastasis of any preceding aspect; methods of increasing the sensitivity of a cancer in a subject to adoptive T cell therapy of any preceding aspect; methods of treating, preventing, inhibiting, reducing, and/or ameliorating a cancer and/or metastasis in a subject of any preceding aspect; or methods of increasing the sensitivity of a cancer in a subject to adoptive T cell therapy of any preceding aspect, wherein the cancer is a BRAF inhibitor resistant cancer and wherein the method further comprises administering to the subject a MEK inhibitor (such as, for example Mekinist, trametinib dimethyl, Binimetinib and/or Cobimetinib).

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, and 1F show that PLX4720 treatment induces a transient up-regulation of M6PR in PLX-sensitive melanoma cells. FIG. 1A shows a typical example of M6PR expression by flow cytometry in two different cell lines (WM983B and WM35) treated for 6 hr with different concentrations of PLX4270. Isot.-cells stained with isotype control. FIG. 1B shows expression of M6PR on the surface of WM983B and WM35 melanoma cells measured by flow cytometry. Geometric mean was calculated and results were normalized to control (DMSO treated samples). Individual results and SD are shown. P values are shown in unpaired twotailed Student's t test. FIG. 1C shows that WM983B cells were treated with DMSO or 10 μM PLX4720 for 3-24 hours in vitro under normoxia and hypoxia (0.5% O2). Cell surface M6PR levels were determined by flow cytometry. Geometric mean was calculated and results were normalized to control (DMSO treated samples under normoxia). Individual results and SD are shown. P values are shown in unpaired twotailed Student's t test. FIG. 1D shows IHC staining of WM35 tumors with human M6PR specific antibody 3 and 5 days after start of the treatment with PLX4720. Typical example of staining is shown. Scale bar=25 μm. FIG. 1E shows images from each section were analyzed using Nis-Elements Ar, sum density was calculated and results were normalized according to the values of vehicle-treated mice. Individual results and SD are shown. P values are shown in unpaired twotailed Student's t test. FIG. 1F shows IHC staining of WM35 tumors with human M6PR specific antibody 3-9 days after finish of the treatment with PLX4720. Typical example of staining is shown. Scale bar=25 μm.

FIGS. 2A, and 2B show temporal regulation and the effect of combination of B-raf inhibitor on M6PR expression. FIG. 2A show cell surface M6PR levels on indicated cell lines after 24 hours treatment with PLX4720 and results were normalized to control (DMSO treated samples). Bars represent standard deviation (SD). Statistical analysis by unpaired two-tailed Student's t test. FIG. 2B shows that WM35 cells were treated with PLX4720 for indicated time. M6PR expression was evaluated by flow cytometry. Results of individual experiments, mean and SD are shown. P values were calculated using two-sided Student's t-test.

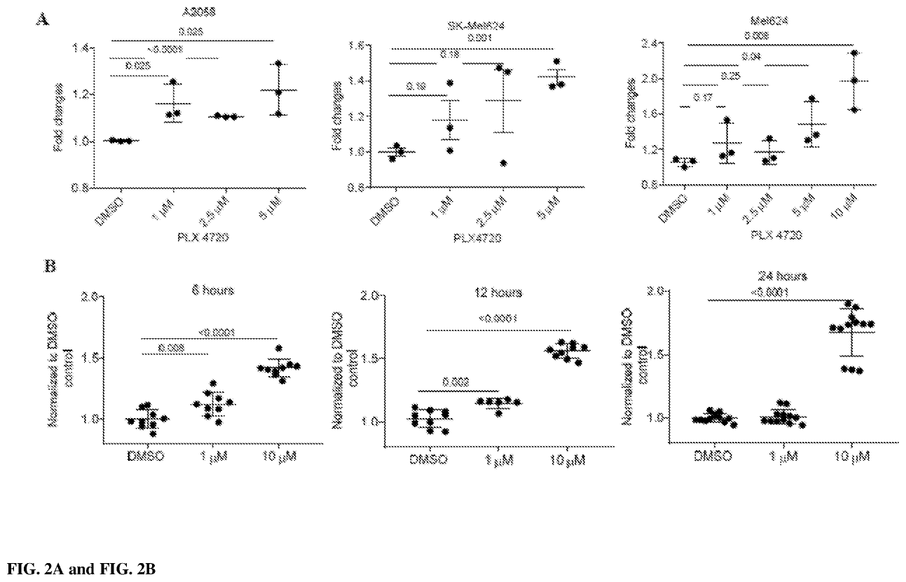

FIGS. 3A, 3B and 3C show the effect of combination of B-raf and MEK inhibitors on M6PR expression. FIG. 3A shows MTT assay results showing the cell percentage of live WM35 cells after treating with vehicle (DMSO) or different doses of Trametinib for 1-4 days. FIG. 3B shows cell surface M6PR levels of WM35 cells, detected after 24 hours treatment of DMSO, PLX4720 only, Trametinib only or combination of PLX4720 and Trametinib by flow cytometry. Geometric mean was calculated and results were normalized to control (DMSO treated samples). Combined results of 3 different experiments. Bars represent standard error mean (SEM). Statistical analysis by unpaired two-tailed Student's t test with significance determined at *p<0.05, ****p<0.0001 versus DMSO treated control group and **p<0.01, ***p<0.001, ****p<0.0001 versus 10 μM PLX4720 treated group. FIG. 3C shows BRAFi (BR) and combined BRAFi+MEKi resistant (CR) cell lines were established by long term exposure to PLX4720 or PLX4720 and Trametinib. M6PR expression was measured by flow cytometry in experimental replicates. Mean and SD are shown. P values in Student's t-test are shown.

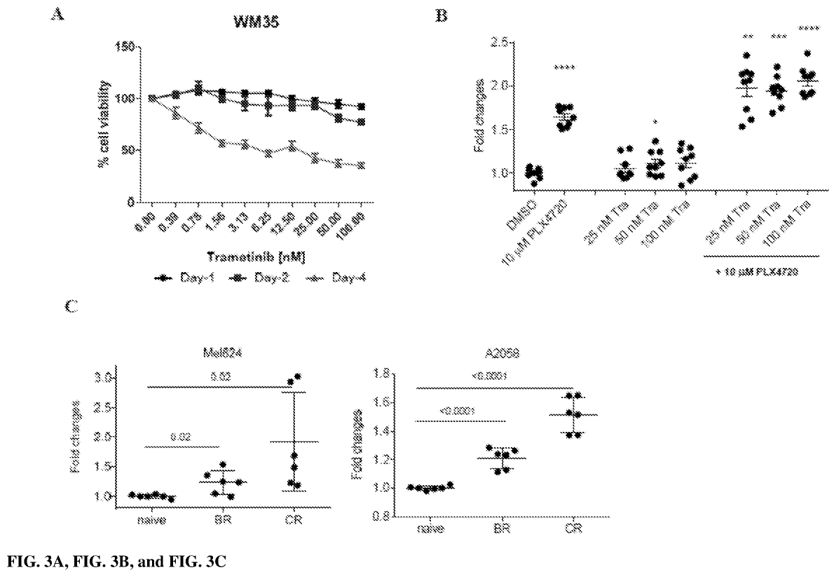

FIGS. 4A and 4B show WM35 cells resistant to PLX4720. FIG. 4A shows BRAFi resistant WM35 cell line (WM-35BR) was established by long term exposure to PLX4720. Cell viability was measured in experimental replicates in MTT test after 2 and 4 days of treatment. Mean and SD are shown. FIG. 4B show the effect of Dabrafenib on the expression of M6PR. Indicated cells were treated with dabrafenib for 16 hr (1 μM or 5 μM) and M6PR expression was evaluated by flow cytometry. Mean and SD are shown. P values were calculated using two-sided Student's t-test; *-p<0.05; ** p<0.01; ***-p<0.001; ****-p<0.0001 from DMSO treated controls.

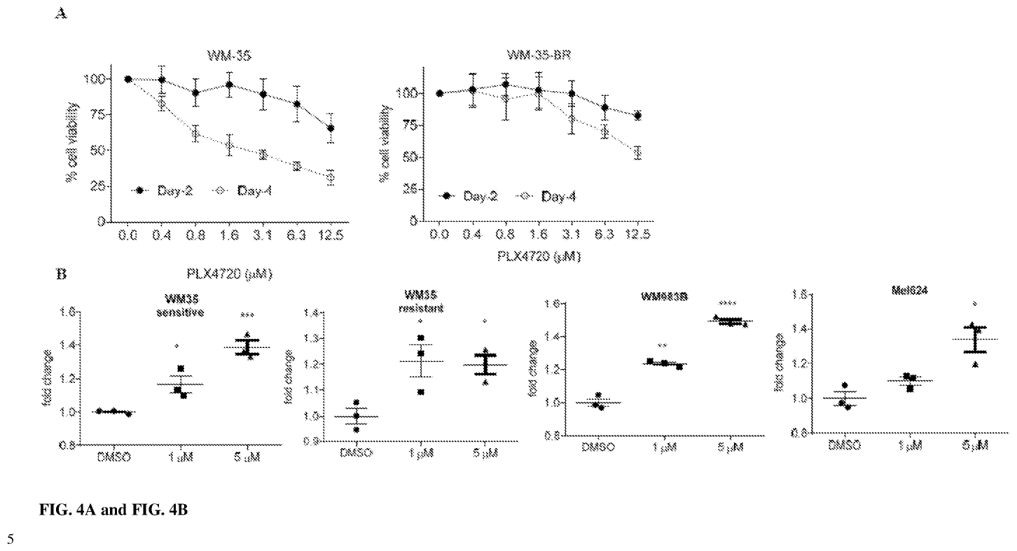

FIGS. 5A, 5B, 5C, 5D, and 5E show that PLX4720 treatment induces a transient up-regulation of M6PR in PLX-resistant melanoma cells. FIG. 5A shows cell surface M6PR after 1 mM and 10 mM PLX4720 treatment in WM983B-BR and WM35-BR cells, respectively. Individual results and SD are shown. P values are shown in unpaired twotailed Student's t test. FIG. 5B shows WM983B-BR cells were treated with DMSO or 10 mM PLX4720 for 6-24 hours in vitro under normoxia and hypoxia. Cell surface M6PR levels were determined by flow cytometry. Geometric mean was calculated and results were normalized to control (DMSO treated samples under normoxia). Individual results and SD are shown. P values are shown in unpaired twotailed Student's t test. FIG. 5C shows IHC staining of WM35-BR derived tumor sections displaying MPR levels after 5 days of PLX4720 treatment of mice. Typical example of staining is shown. Scale bar=25 μm. FIG. 5D shows 10 images from each section were analyzed using Nis-Elements Ar, sum density was calculated and results were normalized according to the values of vehicle (DMSO)-treated mice. Individual results and SD are shown. P values are shown in unpaired twotailed Student's t test. FIG. 5E shows cell surface M6PR on WM35-BR cells after treatment for 24 hours with DMSO, PLX4720, Trametinib or combination of PLX4720 and Trametinib treatment. Geometric mean was calculated and results were normalized to DMSO control. Individual results and SD are shown. P values are shown in unpaired twotailed Student's t test.

FIGS. 6A, 6B, 6C, 6D, 6E, 6F, 6G, and 6H show M6PR up-regulation on the cell surface sensitizes WM35 cells to the cytotoxic effect of TIL. FIG. 6A shows that after pretreatment of target cells (WM35) with PLX4720 O/N, 4-8 hours Cr release assay was performed in triplicate. Cells were labeled with ⁵¹Cr and cultured with effector cells (HLA-A2+TIL) at the indicated ratios. Appropriate maximum and minimum release controls were determined in each experiment. Representative results of 3 different experiments are shown. FIG. 6B shows analysis of total protein from control (pAHygCMV2) or M6PR over-expressing (M6PR/IGF2R) WM35 cells. Lysates were probed with M6PR and HSP90 specific antibodies. FIG. 6C shows cell surface levels of M6PR was determined by flow cytometry in control (WM35-pAHygCMV2) and M6PR-overexpressing cells (WM35-IGF2R/M6PR). Bars represent standard deviation (SD). Statistical analysis was done using unpaired twotailed Student's t test. FIGS. 6D and 6E shows 6 hours ⁵¹Cr experiment was performed in triplicates. As target cells, ⁵¹Cr labeled WM35-pAHygCMV2 (control) and WM35-IGF2R/M6PR were used and cultured with HLA-matching TIL (6D) or healthy donor derived CD8+ T cells (6E). FIG. 6F shows cells were incubated with inactive GrzB for 1 hr and intracellular GrzB level was measured by flow cytometry. Mean and SD of individual experiments are shown (n=6). P values were calculated in two-sided Student's t-test. FIGS. 6G and 6H show that WM35 cells were treated with DMSO or PLX4720 O/N and cell surface levels of M6PR was detected by flow cytometry (6G). FIG. 6H shows GrzB uptake by WM35 cells were detected by intracellular GrzB staining using GrzB specific mouse anti-human antibody. Geometric mean was calculated and all results were normalized to control (DMSO-treated cells). Individual results and SD are shown. P values are shown in unpaired twotailed Student's t test.

FIGS. 7A and 7B show the effect of M6PR overexpression and deletion on melanoma cell sensitivity to PLX4720 in MTT test. FIG. 7A shows M6PR overexpressing cells. Ten experimental replicates were performed 2 and 4 days after start of the treatment. Mean and SD are shown. FIG. 7B shows MTT assay results showing the cell percentage of live WM983B and WM983B-M6PR KO cells after treating with vehicle or different doses of PLX4720 for 1-4 days. Five independent replicates with the same results were performed.

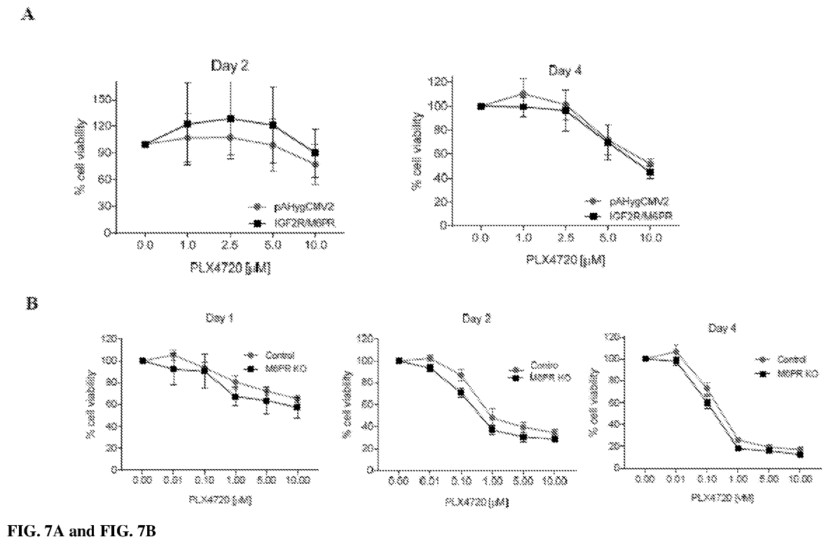

FIGS. 8A, 8B, 8C, 8D, 8E, and 8F show the effect of M6PR deletion on TIL mediated killing of melanoma cells. FIGS. 8A and 8B show expression of M6PR in cell lysates of control (WM983B) and M6PR-deleted (WM983B-M6PR KO) cells tested by western blot (8A) and on the surface of the cells by flow cytometry (8B). Representative data with triplicate for both groups are shown. P values are shown in unpaired twotailed Student's t test. FIG. 8c shows M6PR expression on the cell surface after O/N treatment with DMSO or PLX4720 on WM983B and WM983B-M6PR KO cells. Individual replicates, mean and SD are shown. P values were calculated in unpaired two-sided Student's t test. FIG. 8D shows intracellular GrzB levels were detected by flow cytometry in DMSO and PLX4720 treated cells. Individual results and SD are shown. P values are shown in unpaired twotailed Student's t test. FIG. 8E shows ⁵¹Cr-release cytotoxicity assay was performed in triplicates with TIL and indicated target cells. FIG. 8F shows ⁵¹Cr-release assay performed with TIL and indicated target cells treated ON with PLX-4720. Typical example of three independent experiments is shown. Mean, SD and p values (* p<0.05 in unpaired Student's t-test) are shown.

FIGS. 9A and 9B show the expression of surface molecules on melanoma cells with manipulated expression of M6PR. FIG. 9A shows M6PR-KO WM983B cells. FIG. 9b shows M6PR overexpressing WM35 cells. Appropriate control was used for each cell line as described in the manuscript. Fold increase over control in independent experiments are shown. Cells were seeded in tissue culture plates and 2 day later (80˜90% confluent), cells were collected for staining. Mean and SD of biological replicates (n=5) are shown.

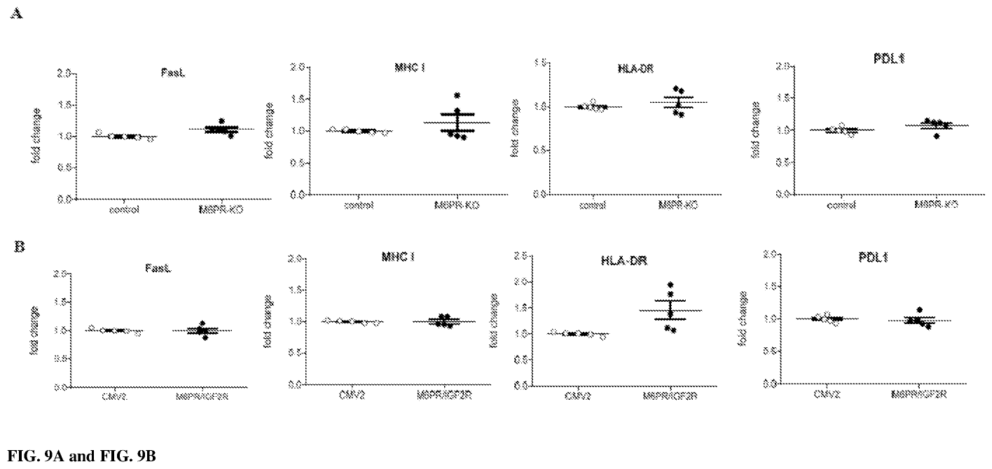

FIGS. 10A, 10B, 10C, 10D, 10E, 10F, 10G, and 10H. The anti-tumor effect of combined therapy. FIG. 10A shows intracellular GrzB level in WM35-BR cells after O/N treatment with DMSO or PLX4720. Individual results and SD are shown. P values are shown in unpaired twotailed Student's t test. FIG. 10B shows left panel-expression of M6PR in cell lysates (parental (P; WM983B-BR), control (C; WM983B-BR-pAHygCMV2) and M6PR over-expressing (M; WM983BBR-M6PR/IGF2R)). Right panel-M6PR expression on cell surface. FIG. 10C shows Grz B uptake by M6PR over expressing WM983B-BR cells were determined by intracellular Grz B staining. Cumulative data of 3 separate experiments are shown. Geometric mean was calculated and all results were normalized to DMSO-treated control. Individual results and SD are shown. P value is shown in unpaired twotailed Student's t test. FIG. 10D shows MTT assay results displaying the percentage of live control and M6PR overexpressing WM983B-BR cells after 2 and 4 days of PLX4720 treatment, respectively. Cumulative data of 2 separate experiments are shown. FIG. 10E show tumor growth kinetics in NSG mice fed with either control diet or a special diet supplemented with 200 mg PLX4720/kg. Each group has 5 mice. Mean and SD are shown. FIG. 10F shows 6 hours ⁵¹Cr release assay performed in triplicates. As target, ⁵¹Cr labeled DMSO (control) or PLX4720 treated WM35-BR cells were cultured with HLA-matching TIL (effector cells) at the indicated ratios. Appropriate maximum and minimum release controls were set up in each experiment. Typical example of 4 different experiments is shown. FIG. 10G shows tumor growth kinetics in WM35-BR bearing NOD/SCID mice treated with PLX4720 and TIL. Arrows indicate TILs injection. PLX4720 was started 5 days before TIL injection and stopped after second TIL injection. Each group has 8 mice. Mean and SEM are shown. P-values were calculated in two-way ANOVA test with Bonferroni-Dunn analysis. FIG. 10H shows tumor weight on day 30-post TIL injection. Individual results and SD are shown. P values are shown in unpaired twotailed Student's t test.

FIGS. 11A, 11B, and 11 C show M6PR expression in patients treated with vemurafenib plus ACT with TIL. FIG. 11A shows PDX were established from four patients who either treatment naïve (4237, 3929), or progress on BRAFi (4070) or combination of BRAFi and MEKi (4298). Mice with PDX were left untreated for 4 weeks (untreated), or treated for 3 weeks with corresponding single agent BRAFi (PLX-PLX4720) or combination with MEKi (CPLX-PLX4720 and PD0325901). Tissues were stained for M6PR and intensity of staining was assessed in 8 fields. Typical example of staining (scale bar=25 μm) and statistical analysis are shown. Intensity was normalized to untreated samples. Mean and SD for 8 fields are shown. P values were calculated in unpaired Student's t-test. FIG. 11B shows Kaplan-Meier progression-free survival and overall survival curves in 16 patients treated with BRAFi and TIL ACT. FIG. 11C shows typical example of M6PR staining in two patients pre- and post-treatment and cumulative results of M6PR staining in 9 patients before and after treatment. H-score results are shown. P value is shown in paired twotailed Student's t test.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder, preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Method of Treating Cancer

Melanoma is a skin cancer with high metastatic potential responsible for 80% of skin cancer-related deaths. Approximately 50% of melanoma patients have the BRAF^{V600E} mutation in their tumors, which leads to expression of constitutively active mutant B-Rapidly Accelerated Fibrosarcoma (BRAF) protein and induces the activation of downstream mitogen activated protein kinase (MAPK) signaling by phosphorylating MEK. Therefore, targeting of BRAF and MEK is an important therapeutic option for BRAF^{V600E} mutated melanoma patients. BRAF inhibitors (BRAFi) vemurafenib and dabrafenib demonstrated impressive clinical responses in patients with BRAF^{V600E} mutant melanoma. Subsequent trials showed that the combination of BRAFi and MEKi achieved higher response rates and greater progression-free and overall survival. However, the efficacy of the treatment is limited due to development of resistance. Several studies have proposed a possible effect of BRAFi on immune responses. A significant increase in the infiltration of CD4+ and/or CD8+ T cells has been shown in metastatic melanoma patients treated with BRAFi. BRAFi increased T cell recognition of melanoma cells without affecting the viability or function of lymphocytes, suggesting that it might increase the effect of immunotherapy. BRAF^{V600E} mutant SM1 melanoma-bearing mice treated with BRAFi and adoptive T cell transfer showed stronger antitumor responses and improved survival compared to either therapy alone. Expression of MHC and tumor antigen by SM1 tumor cells was not significantly altered.

Adoptive cell therapy (ACT) of melanoma with tumor-infiltrating lymphocytes (TIL) derived from patients' resected tumors has demonstrated therapeutic promise. The combination of targeted therapy and ACT would be a natural choice. In a recent pilot trial, the combination of vemurafinib and TIL ACT showed acceptable toxicity and generated objective clinical responses. However, the mechanism of a possible combined effect remains unclear since recognition of autologous tumor by T cells was similar between TILs grown from pre- and post-vemurafenib metastases. The clinically relevant question remained whether the combination of BRAFi and ACT could be beneficial in patients who developed resistance to BRAFi and MEKi and for whom clinical options are very limited.

We have previously demonstrated that transient up-regulation of cation-independent mannose 6-phosphate receptor (M6PR) (also known as insulin-like growth factor 2 receptor; IGF2R) was important for the antitumor effect of combination immune- and chemo- or radiation therapy in different mouse models of cancer. M6PR is a multifunctional membrane-associated protein involved in trafficking of soluble lysosomal proteins in the cytoplasm and binding of M6P containing ligands, such as insulin-like growth factor 2 (IGF2). Importantly, it is a receptor for granzyme B (GrzB) secreted by activated cytotoxic T cells (CTL). Chemotherapy and radiation therapy caused autophagy of tumor cells that resulted in re-distribution of M6PR to the surface of tumor cells and increased uptake of GrzB released by CTLs leading to expansion of tumor cell death. We asked whether BRAF targeted therapy can induce similar effects in human melanoma, and more importantly, whether this effect depends on the development of BRAF resistance by tumor cells. Importantly, as shown herein, it was confirmed that BRAF inhibition induces higher expression of M6PR in melanoma cells in culture. Importantly, this effect was also seen in BRAFi-resistant cells. Furthermore, increased expression of M6PR correlated with higher intake of granzyme B and increased sensitivity of melanoma cells to the toxic activity of tumor infiltrating lymphocytes. Accordingly, in one aspect, disclosed herein are methods of treating, preventing, inhibiting, reducing, and/or ameliorating a cancer and/or metastasis in a subject using an adoptive T cell therapy and/or or methods of increasing the sensitivity of a cancer in a subject to adoptive T cell therapy, the method comprising administering to the subject a BRAF inhibitor (such as, for example, sorafenib, vemurafenib, dabrafenib, and/or encorafenib) and an adoptive T cell therapy, wherein administration of the BRAF inhibitor increases insulin-like growth factor II receptor (IGF2R).

Also disclosed herein are methods of treating, preventing, inhibiting, reducing, and/or ameliorating a cancer and/or metastasis in a subject with an adoptive T cell therapy and/or methods of increasing the sensitivity of a cancer in a subject to adoptive T cell therapy, the method comprising administering to the subject an lGF2R agonist (such as, for example, clenbuterol) and an adoptive T cell therapy.

The methods for treating, preventing, inhibiting, reducing, and/or ameliorating a cancer and/or metastasis in a subject using an adoptive T cell therapy and/or or methods of increasing the sensitivity of a cancer in a subject to adoptive T cell therapy comprising administration of a BRAFi or IGF2R agonist disclosed herein each utilize some form of adoptive T cell immunotherapy. In one aspect, it is understood and herein contemplated that the adoptively transferred T cells for use in the disclosed methods can be chimeric antigen receptor T cells (CAR T cells) and/or tumor infiltrating lymphocytes (TlLs)

As noted throughout this application, it is understood and herein contemplated that the methods and inhibitors disclosed herein for sensitization to ACT/TIL immunotherapies can be used to treat, inhibit, reduce, prevent, and/or ameliorate any disease where uncontrolled cellular proliferation occurs such as cancers (including, but not limited to primary cancers and metastasis). A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer, colon cancer, rectal cancer, prostatic cancer, or pancreatic cancer and tumors comprising mutant BRAF tumor types and treatment resistant tumors. In one aspect, of the tumors disclosed can include other BRAF mutant tumor types to include, but are not limited to colorectal cancer, melanoma, colorectal carcinoma, papillary thyroid carcinoma, hairy cell leukemia, and Langerhans cell histiocytosis, pleomorphic xanthoastrocytoma, ganglioglioma, epithelioid glioblastoma, and gliomas diagnosed at a younger age; melanoma, colorectal, thyroid, and Non-small cell lung cancer (NSCLC), as well as hairy cell leukemia.

In one aspect, it is understood and herein contemplated that the methods disclosed herein are not exclusive to each other. Thus, in one aspect disclosed herein are methods of methods of treating, preventing, inhibiting, reducing, and/or ameliorating a cancer and/or metastasis in a subject using an adoptive T cell therapy and/or or methods of increasing the sensitivity of a cancer in a subject to adoptive T cell therapy, the method comprising administering to the subject a BRAF inhibitor (such as, for example, sorafenib, vemurafenib, dabrafenib, and/or encorafenib) and an adoptive T cell therapy, wherein administration of the BRAF inhibitor increases insulin-like growth factor II receptor (IGF2R) further comprising the administration of an lGF2R agonist (such as, for example, clenbuterol). Similarly, disclosed herein are methods of treating, preventing, inhibiting, reducing, and/or ameliorating a cancer and/or metastasis in a subject with an adoptive T cell therapy and/or methods of increasing the sensitivity of a cancer in a subject to adoptive T cell therapy, the method comprising administering to the subject an lGF2R agonist (such as, for example, clenbuterol) and an adoptive T cell therapy further comprising administering to the subject a BRAF inhibitor (such as, for example, sorafenib, vemurafenib, dabrafenib, and/or encorafenib).

Also disclosed herein are methods of treating, preventing, inhibiting, reducing, and/or ameliorating a cancer and/or metastasis comprising administering a BRAFi; methods of increasing the sensitivity of a cancer in a subject to adoptive T cell therapy comprising administering a BRAFi; methods of treating, preventing, inhibiting, reducing, and/or ameliorating a cancer and/or metastasis in a subject comprising administering to a subject a lGF2R agonist; or methods of increasing the sensitivity of a cancer in a subject to adoptive T cell therapy comprising administering to the subject lGF2R agonist, wherein the cancer is a BRAF inhibitor resistant cancer and wherein the method further comprises administering to the subject a MEK inhibitor (such as, for example Mekinist, trametinib dimethyl, Binimetinib and/or Cobimetinib). Thus, in one aspect, disclosed herein are any combination of a MEK inhibitor (such as, for example Mekinist, trametinib dimethyl, selumetinib, Binimetinib and/or Cobimetinib), a BRAF inhibitor (such as, for example, sorafenib, vemurafenib, dabrafenib, and/or encorafenib), and/or an lGF2R agonist (such as, for example, clenbuterol). Examples of the combination of the MEK inhibitor and BRAF inhibitor can comprise Tafinlar and Mekinist; dabrafenib mesylate and trametinib dimethyl; Encorafenib and Binimetinib; and Vemurafenib and Cobimetinib.

In one aspect, it is understood and herein contemplated that successful treatment of a cancer in a subject is important and doing so may include the administration of additional treatments that may or may not inhibit BRAF or be an lGF2R agonist. Thus, the disclosed treatments can further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine 1131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Where an EGFR splice variant isoform is not detected, the treatment methods can include or further include checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016). Where the presence of an EGFR splice variant isoform is detected the treatment regimen implemented does not include a immune checkpoint blockade inhibitor. It is understood and herein recognized that the presence of an EGFR splice variant isoform does not necessarily indicate that the cancer is resistant to all immune checkpoint blockade inhibitors. In one aspect, the detection of the EGFR splice variant isoform indicates resistance to PD-1, PD-L1, PD-12, CRLA-4, IDO, B7-H3, B7-H4, TIM3, or LAG-3. In one aspect, the detection of the EGFR splice variant isoform indicates resistance to PD-L1. Thus, when resistance is only to a particular form of immune checkpoint blockade inhibition (such as, for example PD-L1), other immune checkpoint blockade inhibitors can still be used.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: BRAF Targeting Sensitizes Resistant Melanoma to Cytotoxic T Cells

a) Material and Methods

(1) Clinical Trial

The clinical trial protocol (NCT01659151) was approved by institutional review board of University of South Florida, and all subjects gave written informed consent for trial participation. The studies were conducted in accordance Declaration of Helsinki guidelines. Subjects were of age ≥18 years with stage III or IV metastatic melanoma that harbored an activating BRAF V600 mutation and were determined to be unresectable for intent to cure. Existing CNS metastases were required to be treated unless three or less in number, each less than 1 cm in size, and none associated with hemorrhage/edema. A focus of at least 1 cm of metastatic melanoma was harvested for TIL propagation with residual measurable disease per RECIST 1.1 criteria. Subjects started vemurafenib 960 mg (supplied by Genentech, Inc) by mouth twice daily one day following surgical resection with allowance for dose reduction or cessation based upon intolerance per the standard of care. TIL was expanded from tumor fragments and evaluated for reactivity, with the exception of the final two patients where rapid expansion occurred in G-REX flasks due to change in manufacturing requirements. Upon successful TIL propagation, subjects underwent lymphodepleting chemotherapy comprising daily inpatient cyclophosphamide 60 mg/kg×for 2 days, followed by daily outpatient fludarabine 25 mg/m2 for 5 days. Subjects then underwent ACT followed by up to 15 doses of intravenous 720,000 IU/kg of Interleukin-2 every 8 hours in the hospital under telemetry monitoring. Following clinical recovery from ACT, subjects were discharged and resumed vemurafenib for up to two years or until disease progression or intolerance.

Pre and post-treatment tumor biopsies were obtained from 9+7=16 patients receiving therapy for metastatic melanoma, either as part of a clinical trial of vemurafenib treatment prior to and after adoptive cell therapy for metastatic melanoma (n=9). Patients underwent needle core biopsies of target lesions after receiving therapy, and pre-treatment tumor tissue was retrieved from the institutional archives for comparative staining.

(2) Human Cells and Mouse Models

Human studies were approved by The Wistar Institute IRB. Peripheral blood was collected from healthy volunteers after obtaining informed consent. Animal experiments were approved by The Wistar Institute Animal Care and Use Committee. The mice were kept under pathogen free conditions. Experiments were carried out using female NSG mice or female and male nonobese diabetic/severe combined immunodeficient (NOD/SCID) mice, obtained from the mouse facility of the Wistar Institute and Charles River Laboratories, respectively. WM35 human melanoma and HLA-matched human tumor infiltrating lymphocytes (TIL) were generated at H. Lee Moffitt Cancer Center and Research Institute, Tampa, Fla., USA). Human melanoma cells lines WM983B and WM983B-BR were maintained in Dulbecco's Modified Eagles medium (DMEM) (Gibco), other human melanoma cell lines were maintained in RPMI medium supplemented with 10% fetal bovine serum (FBS, Sigma-Aldrich, St. Louis, Mo.) and 1% Pen/Strep (Corning; Cat. no. 30-002-Cl) at 37° C., 5% CO2. PLX4720-resistant cells were kept in cell culture media supplemented with 1 μM PLX4720 to keep these cells resistant against the drug. 350 μg/mL Hygromycin B (Invitrogen; Cat. no. 10687010) was kept in the culture media of WM983B-BR-pAHygCMV2, WM983B-BR-M6PR/IGF2R, WM35-pAHygCMV2, WM35-M6PR/IGF2R cells after transfection. Tumor cell lines were tested for mycoplasma contamination by using Universal Mycoplasma detection kit (ATCC) regularly. Human melanoma cells were treated with 1-10 μM PLX4720 (Selleckchem; Cat. no. S1152), 25-100 nmol/mL (nM) Trametinib (Selleckchem; Cat. no. S2673). Hypoxia (0.5% 02) was maintained using hypoxic chamber (BioSpherix).

In order to establish xenograft models, cultured WM35, WM35-BR, WM983B-BR-pAHygCMV2 and WM983B-BR-M6PR/IGF2R cells were harvested, suspended in sterile PBS, mixed in 2:1 ratio with Matrigel and injected subcutaneously into the left flank of mice (1-3×10⁶ cells per mouse). PLX4720 was administered daily at dose 50 mg/kg for 10 days via oral gavage. As a control, mice treated with vehicle alone (2% DMSO, 50% PEG 300, 5% Tween 80) were used. Treatments were started when tumors were 0.5-0.8 cm in diameter.

(3) Generation of Human Melanoma Cells Overexpressing Human M6PR/IGF2R

The plasmid pAHygCMV2/IGF2R encoding wild-type human M6PR/IGF2R was kindly provided by Dr. Lukas Mach from Medical University of Vienna. WM983B-BR and WM35 cell lines were stably transfected with either an empty plasmid, pAHygCMV2, or pAHygCMV2/IGF2R using Lipofectamine™ LTX reagent (Thermo Fischer Scientific; Cat. no. 15338030). Selection of transfected cells was achieved by their ability to grow in the presence of 350 μg/ml Hygromycin B (Invitrogen). Drug-resistant clones were isolated approximately after 2 weeks and tested for M6PR/IGF2R production by flow cytometry and western blotting.

(4) Generation of WM983B-M6PR KO Tumor Cell Line Using CRISPR/Cas9

Human M6PR/IGF2R gene (gene ID number 3482) was used as target to design DNA guides. Oligoduplexes were ligated to the vector pLentiCRISPRv2 and transformed to Stbl3 bacteria. pLentiCRISPRv2 vectors containing the sgRNA guides were transfected with pMD2.G and pSPAX2 to 293T cells in order to produce lentiviral vectors. From all six guides generated, AGTCCGGGCCCGGCGCGATG (SEQ ID NO: 1) gave a polyclonal population that included clones with complete knock out of M6PR. Sixteen clones were isolated by cell serial dilution and in seven of them, the gene had been deleted.

(5) Immunohistochemistry of Patients Samples

Slides were stained using a Ventana Discovery XT automated system (Ventana Medical Systems, Tucson) as per manufacturer's protocol with proprietary reagents. Antibody sources, dilutions, and incubation times were as follows: 1. CD4 rabbit anti-human #104R-18, Cell Marque, Rocklin, Calif., prediluted, incubation for 8 minutes 2. CD8 rabbit anti-human #790-4460, Ventana, Tucson, Ariz., prediluted, incubation for 20 minutes 3. Granzyme rabbit anti-human #760-4283, Ventana, Tucson, Ariz., prediluated, incubation for 44 minutes 4. Mannose-6 phosphate receptor rabbit anti-human #ab32815, Abcam, Cambridge, Mass., 1:1000 dilution, incubation 32 minutes. The Ventana UltraMap Anti-rabbit Alk Phos secondary Antibody was used for 8 minutes (CD4) and 16 minutes (CD8. Granzyme B, mannose-6 phosphate receptor). The detection system used was the Ventana ChromoMap Red kit and slides were then counterstained with hematoxylin. Stained slides were evaluated and graded by the study pathologist. For CD4 and CD8, the density of intratumoral lymphocytes was graded from 0-3 as follows: 0—absent, 1—rare lymphocytes, 2—lymphocytes scattered singly and in small aggregates, 3—dense infiltrate of lymphocytes. For mannose-6 phosphate receptor and Granzyme B, which both exhibited cytoplasmic staining, the staining was graded from 0-3 as follows: 0—no staining, 1—weak staining, 2—moderate staining, 3—strong staining.

(6) Immunohistochemistry of Tumor Tissues in Xenograft Experiments

Tumor tissues were harvested, fixed overnight with 4% paraformaldehyde (Electron Microscopy Science; Cat. no. 15710) at 4° C., embedded in paraffin blocks and 4-5 μm thick sections of the tissue were prepared and stained with polyclonal goat M6PR/IGF2R primary antibody (R&D Systems; Cat. no. AF2447; 5 μg/mL per samples) O/N at 4° C. Biotinylated 2nd antibody in 1:200 dilution was used for 30 min at RT followed by application of ABC solution (avidin dehydroxygenase and biotinylated horseradish peroxidase) for 30 min at RT. DAB 0.05% solution was applied for 3-4 min at RT. Hematoxylin was used for counterstaining the nucleus. Images were analyzed using Nis-Elements Ar (Advanced Research) Nikon 80i Upright Microscope with 40×N.A. objective. Each slide was scanned, images were manually captured at separate X, Y locations and a multipoint ND document was created point by point. A threshold value was defined for each image (for brown M6PR staining) using the single point selection tool. Results were exported to Excel for further analysis. (Acquisition Software: Nikon NIS-Elements Br (Basic research) 4.0, Camera Name: DS-Ri1-U3 40× Objective, Numerical Aperture: 0.95, Camera Settings: Format: 1280×1024 Fine, Exposure: ME 80 ms (−+0.0 EV))

(7) MTT Assay

CellTiter 96@ Non-reactive cell proliferation assay kit (Promega; Cat. no. G4000) was used to detect viability of the cells and experiments were performed according to the manufacturer's protocol.

(8) Flow Cytometry

M6PR expression levels were detected by flow cytometry. Cells were incubated with Aqua dead cell staining kit (Thermo Fischer Scientific; Cat. no. L34957) for 15 min at 4° C., followed by staining with Alexa Fluor 647-conjugated mouse antibody specific for human IGF2R/M6PR (BD Biosciences; Cat. no. 565105; clone no. MEM-238) or its isotype (BD Biosciences; Cat. no. 557732; clone no. MOPC-21) at 4° C. for 20 minutes. Cells were fixed using 1% paraformaldehyde for 20 minutes at 4° C. before running them on LSR14. Data was analyzed by FlowJo software (Tree Star).

(9) Granzyme B (GrzB) Uptake

Melanoma cells were were incubated with inactive granzyme B (R&D systems; Cat. no. 2906-SE-010, Stock concentration: 0.22 mg/mL, Lot: OFN0415101) at 37° C., 5% CO2 for 1 hour. Cells were then stained with Aqua dead cell staining kit (Thermo Fischer Scientific) for 15 min at 4° C. fixed in fixation buffer (BD Cytofix/Cytoperm; Cat. no. 51-2090). Granzyme B antibody (Cat. no. 561142) or its isotype control (BD Bioscience; Cat. no. 555749) were used. Stained cells were run on LSR14. Data was analyzed by FlowJo software (Tree Star).

(10) Mini Rapid Expansion Protocol (Mini-REP)

In a T25 flask, 1.45×10⁵ human TIL were stimulated with 30 ng/mL CD3 monoclonal antibody

(OKT3) (eBioscience; Cat. no. 16-0037-81) in the presence of 29×10⁶ irradiated (5000 rad) allogenic PBMC as feeder cells. TIL were cultured in 9.6 mL of REP Media I (comprised of RPMI 1640, 10% heat-inactivated human AB serum (Sigma-Aldrich; Cat. no. H4522), 55 μM 2-mercaptoethanol (Gibco; Cat. no. 21985-023), 10 mM HEPES Buffer (Corning; Cat. no. 30-060-C1)) and 10.7 mL AIM V (Gibco; Cat. no. 0870112-DK) supplemented with 6000 I.U./mL rhIL-2. On day 4.15 mL media was replaced with fresh media (50% AIM V and 50% REP I) containing 3000 I.U./mL rhIL-2. On day 7.15 mL media was removed and 15 mL of AIM V containing 3000 I.U./mL rhIL-2 was added to the culture. On day 9, TIL and media were transferred T25 to T75 flask and 20 mL of AIM V containing 3000 I.U./mL rhIL-2 was added. On Day 11, TIL and media were transferred from T75 to T150 flask and 40 mL of AIM V containing 3000 I.U./mL rhIL-2 was added. After 14 days, TIL were collected, counted and CD8+ TIL were isolated using EasySep™ Human CD8+ T Cell Enrichment Kit (Stem Cell Technologies; Cat. no. 19053) for further experiments.

(11) CTL Assay

For Chromium (⁵¹Cr) release assays, 1×10⁶ human melanoma cells were incubated with 100 μCi ⁵¹Cr (Perkin-Elmer; Cat. no. NEZ030S001MC; 1 mCi/mL) at 37° C. for 60 minutes, washed 3 times with sterile PBS and plated into 96-well round-bottom plates at a cell density of 1×10⁴ tumor target cells/well. Target cells were incubated with human CD8+ TILs in triplicates at the indicated effector/target (E) ratios in 200 μl culture medium at 37° C. in a humidified CO2 incubator. After incubation, plates were centrifuged, 50 μl supernatant was harvested from each well and ⁵¹Cr release was measured using a gamma counter. The percent specific lysis was calculated as follows: 100×[(experimental release−spontaneous release)/(maximum release−spontaneous release)]. As controls, T cells isolated from blood of healthy donor were used.

(12) Western Blotting

Cells were lysed in RIPA buffer (Sigma-Aldrich) in the presence of protease inhibitor cocktail (Sigma-Aldrich). Whole cell lysates were subjected to 6% SDS-PAGE and transferred to PVDF membrane. The membranes were probed with the antibodies specific for M6PR (Cell Signaling Technology; Cat. no. 14364) and HSP90 (Cell Signaling Technology; Cat. no. 4877) and secondary antibody conjugated with peroxidase (Santa Cruz; Cat. no. sc-2357).

(13) Statistical Analysis

P values were determined by 2-tailed student's t test (unpaired). For repeated measurements two-way ANOVA test followed by the Bonferroni-Dunn method was used. All calculations were performed on GraphPad Prism7. All results are presented as mean±SD or ±SEM (*p<0.05; **p<0.01; ***p<0.001; ****p<0.0001). Responses and survival of subjects treated on the clinical trial were determined by RECIST 1.1 criteria and by the Kaplan-Meier method using GraphPad Prism7 respectively.

b) Results

(1) BRAF Inhibition Causes Up-Regulation of M6PR in Human Melanoma Cell Lines

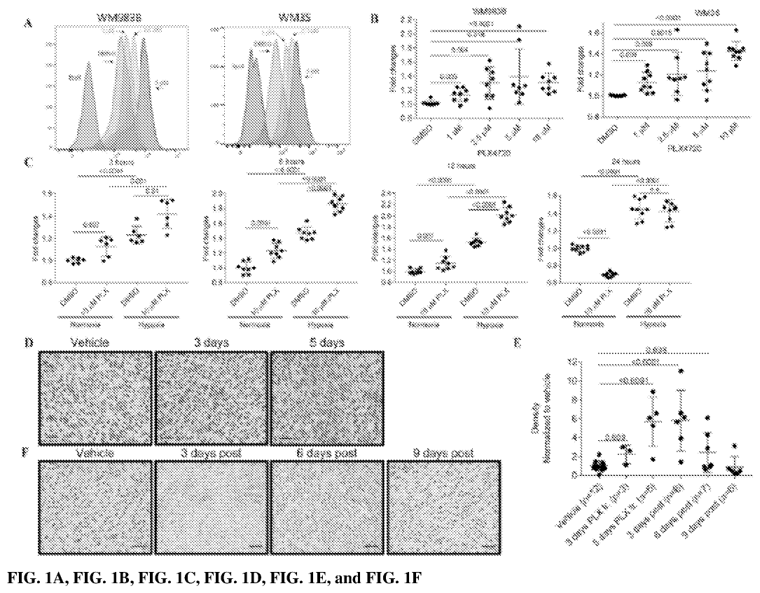

We tested the effect of the BRAFi PLX4720 on M6PR expression using BRAF^{V600E} mutant human melanoma cell lines WM983B and WM35. Within 6 hours of treatment with 1 μM, 2.5, 5 or 10 μM PLX4720, both melanoma cell lines showed substantial dose-dependent up-regulation of M6PR on the cell surface (FIG. 1A,B). Similar up-regulation of M6PR was also detected in other melanoma cell lines (A2058, SK-Mel624, Mel624).

Since hypoxia is an important component of the tumor microenvironment, we evaluated the effect of hypoxia on PLX4720-induced M6PR up-regulation. WM983B melanoma cells were exposed to 0.5% 02 during PLX4720 treatment. Within 3 hours after starting treatment, a significant (p<0.0001) increase in the expression of M6PR during hypoxia was detected (FIG. 1C). M6PR up-regulation upon PLX4720 treatment was more pronounced under hypoxic conditions than in normoxia and reached a maximum within 12 hours. Twenty-four hours after starting treatment no up-regulation of M6PR expression by PLX4720 was detected (FIG. 1C). Similar kinetic of M6PR up-regulation was observed in WM35 cells. To understand the effect of PLX4720 on M6PR expression in vivo, WM35 tumor cells were injected s.c. into immune deficient NOD/SCID mice. When tumors reached 0.5-0.8 cm in diameter, mice were treated with vehicle or 50 mg/kg PLX4720 by oral gavage for 3 or 5 consecutive days. A significant increase in tumor M6PR levels was detected by immunohistochemistry 3 days after start of the treatment and was further increased 2 days later (FIG. 1D, E). Expression of M6PR remained high 3 days after finish of the treatment with slight decrease by day 6. M6PR expression returned to the pretreatment level by day 9 after finishing the treatment (FIG. 1E, F).

Combination BRAF and MEK inhibitors significantly increases clinical responses and survival in metastatic melanoma patients. We asked whether combining PLX4720 with a MEKi, trametinib, would affect the up-regulation of M6PR. As expected, 4-day in vitro treatment of melanoma cells with trametinib caused a decrease in cell viability. However, in contrast to PLX4720, trametinib did not induce M6PR expression. Treatment of tumor cells with the combination of trametinib and PLX4720 resulted in significantly higher up-regulation of M6PR than PLX4720 alone. Thus, BRAFi caused substantial up-regulation of M6PR on human melanoma cells in vitro and in vivo. In vivo this effect lasted for almost a week after cessation of the treatment.

(2) PLX4720 Treatment Causes Up-Regulation of M6PR in PLX4720-Resistant Human Melanoma Cell Lines

One of the major problems in the treatment of melanoma with BRAFi or BRAFi+MEKi is the development of resistance. We evaluated the effect of PLX4720 on M6PR expression in BRAF resistant cell lines generated by long-term exposure to increased concentrations of inhibitors. Cell lines resistant to BRAFi or to combination of BRAFi and MEKi showed increased (p=0.02) expression of M6PR as compared to untreated cell lines.

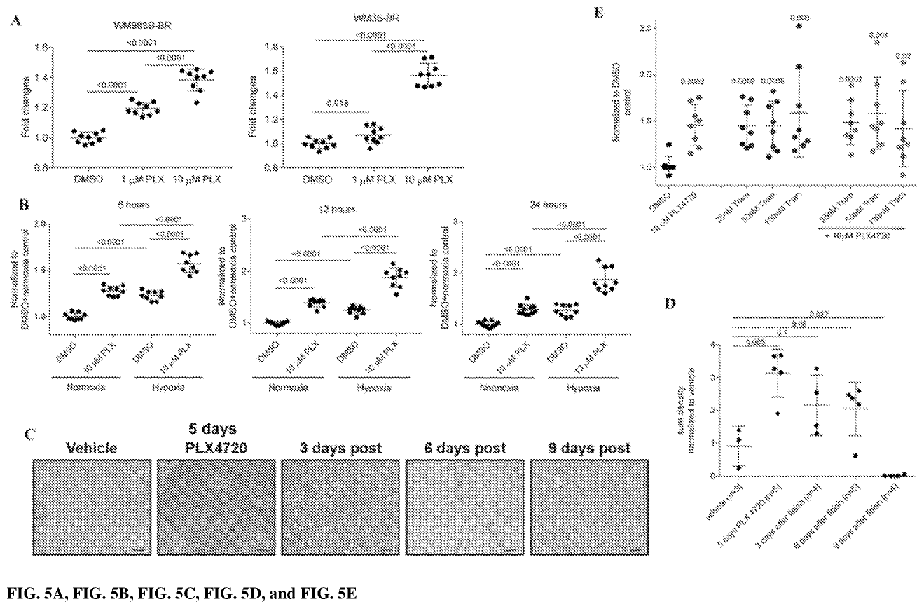

To maintain consistency with the results obtained on sensitive cell lines and to better assess the effect of BRAFi on the sensitivity of tumor cells to CTLs, we generated an additional BRAFi resistant cell line WM35-BR by exposing WM35 cells to increased concentrations of PLX4720. Resistant cell lines (WM983B-BR and WM35-BR) were treated with 1 μM or 10 μM PLX4720 for different times, and cell surface M6PR levels were determined by flow cytometry. Despite the fact that these cells were previously exposed to BRAFi, treatment with PLX4720 caused further substantial up-regulation of MP6R on the cell surface (FIG. 5A). Similar to the effect seen in sensitive cells, we have observed increased M6PR expression in PLX4720-treated BRAF resistant cells under hypoxia (FIG. 5B). However, in contrast to PLX4720-sensitive cells, M6PR levels did not decrease to pre-treatment levels after 24 hours of treatment (FIG. 5B).

We also assessed the effect of the other BRAFi-dabrafenib (GSK2118438) on the expression of M6PR in different melanoma cell lines and observed substantial up-regulation of the receptor similar to the effect seen with PLX4720.

WM35-BR tumor cells were injected subcutaneously to the flank of NOD/SCID mice and once the average tumor area reached around 40-50 mm2 in size, mice were treated with vehicle or 50 mg/kg PLX4720 by oral gavage for 5 days. After 5 days of PLX4720 treatment, tumors were harvested at different time points and M6PR expression in tumor tissues was detected by IHC staining. Five days of PLX4720 treatment caused a marked up-regulation of M6PR in tumor tissues, which was lower than that observed in sensitive cell lines. In contrast to sensitive lines, expression of M6PR in resistant cells decreased more rapidly after treatment was stopped (FIGS. 5C and 5D). We then tested the effect of combined BRAFi and MEKi in PLX4720-resistant WM35-BR cells. In contrast to sensitive cells, in resistant cell lines, trametinib alone significantly up-regulated expression of M6PR and this effect was not further increased by the combination of BRAFi and MEKi (FIG. 5E). Thus, up-regulation of M6PR by BRAFi was observed not only in sensitive, but also in resistant cell lines.

(3) M6PR Up-Regulation Sensitizes Melanoma Cells to the Cytotoxic Effect of Tumor Infiltrating Lymphocytes In Vitro

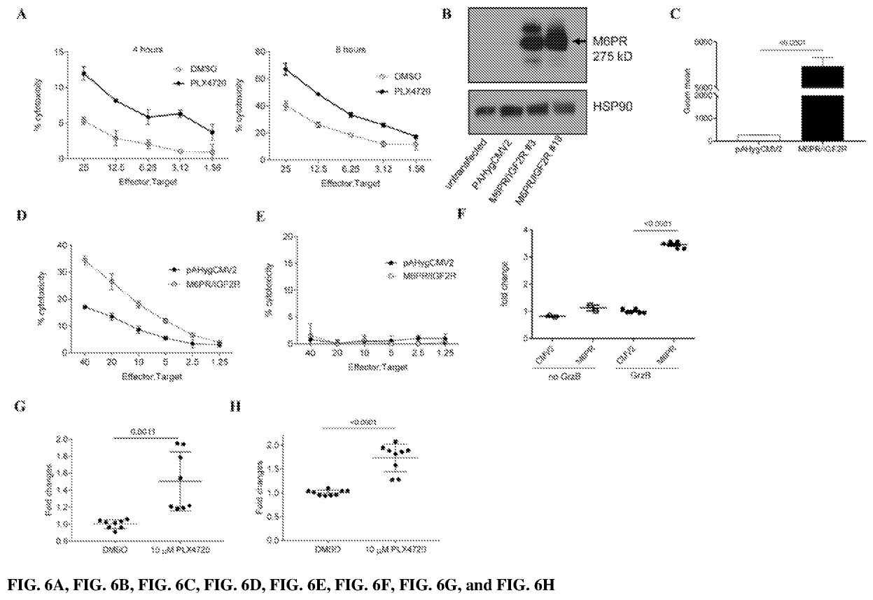

It is explored herein whether BRAF targeting can affect the sensitivity of melanoma cells to CTLs. HLA-A2+ human tumor infiltrating lymphocytes (TIL) that recognized HLA-A2+ matched WM35 tumor cells were obtained from a patient with metastatic melanoma and expanded by a mini rapid expansion protocol. CD8+ T cells were isolated and used as effector cells in a CTL assay. WM35 cells treated with DMSO or PLX4720 were used as targets. We observed that PLX4720 treatment rendered WM35 cells more sensitive to the lysis by effector cells (FIG. 6A). To assess whether M6PR up-regulation can directly enhance cell killing by CTLs, we generated WM35 and WM983B cell lines with stable over expression of M6PR (WM35-M6PR/IGF2R, WM983B-M6PR/IGF2R) (FIGS. 6B and 6C). Overexpression of M6PR did not affect sensitivity of melanoma cells to PLX4720. However, incubation with TIL resulted in a significantly higher killing of M6PR over-expressing than control cells (FIG. 6D). T cells isolated from peripheral blood of healthy individual were not able to kill target cells (FIG. 6E). Granzyme B (GrzB) released by CTLs is one of the ligands of M6PR that can potentiate the cytotoxic effect of CTLs. We evaluated GrzB uptake by melanoma cells with overexpression of M6PR and found dramatically higher GrzB uptake in M6PR overexpressing cells than controls (FIG. 6F).

In order to understand whether BRAF targeting affects GrzB uptake by tumor cells WM35 cells were treated overnight with PLX4720 and M6PR up-regulation was confirmed by flow cytometry (FIG. 6G). Cells were then incubated with inactive recombinant GrzB for an hour and intracellular GrzB was assessed by flow cytometry. PLX4720 treatment caused significant up-regulation of GrzB uptake by tumor cells (FIG. 6H).

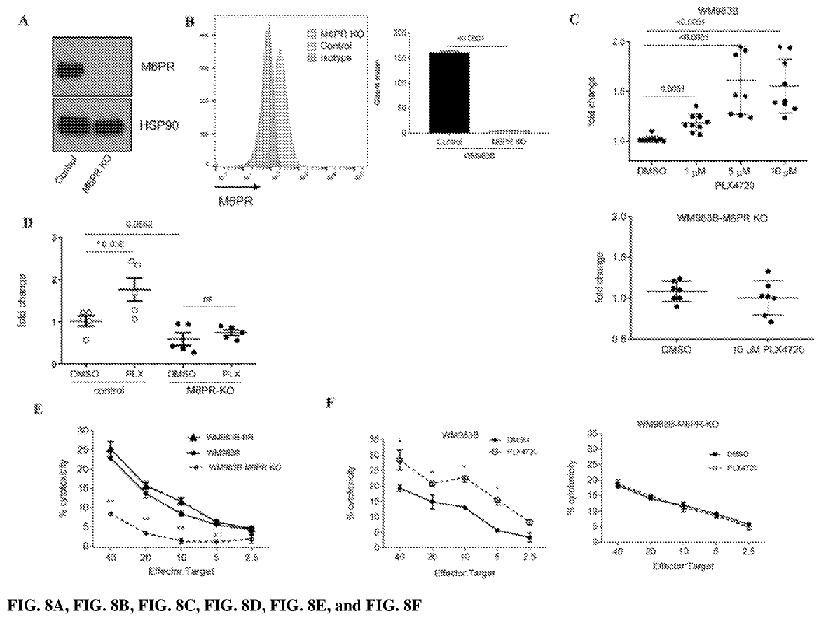

To investigate if M6PR has a direct role in the increased uptake of GrzB induced by PLX4720, we generated a WM983B-M6PR-KO cell line lacking M6PR expression, using Crispr/Cas9 technology. Deletion of M6PR was confirmed by western blotting (FIG. 8A) and flow cytometry (FIG. 8B). WM983B and WM983B-M6PR-KO cells were cultured with DMSO or different concentrations of PLX4720. As expected, PLX4720 induced substantial up-regulation of M6PR in WM983B cells but not in WM983B-M6PR-KO cells (FIG. 8C). Deletion of M6PR did not affect the sensitivity of WM983B tumor cells to PLX4720 treatment. WM983B and WM983B-M6PR-KO cells were treated overnight with DMSO or PLX4720 and then incubated with inactive GrzB for 1 hour at 37° C. and intracellular GrzB levels were measured by flow cytometry. In the absence of M6PR, PLX4720-inducible up-regulation of GrzB uptake was abrogated (FIG. 8D) indicating that PLX4720-induced GrzB uptake depends on the up-regulation of M6PR. To test the sensitivity of WM983B cells to CTLs, we expanded TILs isolated from a HLA-A1+ patient with metastatic melanoma. These TILs recognized HLA-A1+WM983B cells. As targets, we used WM983B and WM983B-M6PR-KO cells. Deletion of M6PR in tumor cells markedly reduced their killing by TILs as compared with WT cells. BRAFi sensitive and resistant WM983B cells were recognized by CTLs equally well (FIG. 8E). Treatment with PLX4720 significantly (p<0.05) increased killing of WM983B tumor cells by TILs, whereas this effect was not observed in WM983B-M6PR-KO cells (FIG. 8F). Herein was asked whether manipulation with expression of M6PR could affect expression of molecules associated with antigen presentation and regulation of immune responses by tumor cells. We evaluated expression of MHC class I, MHC class II, PDL1, and FasL on tumor cells with overexpression or deletion of M6PR. No significant changes were observed in any of those molecules. Taken together, these results indicate that BRAF inhibition sensitized tumor cells to CTLs via up-regulation of M6PR in both sensitive and resistant melanoma cells.

(4) M6PR Up-Regulation Sensitizes PLX4720-Resistant Melanoma Cells to the Cytotoxic Effect of TIL

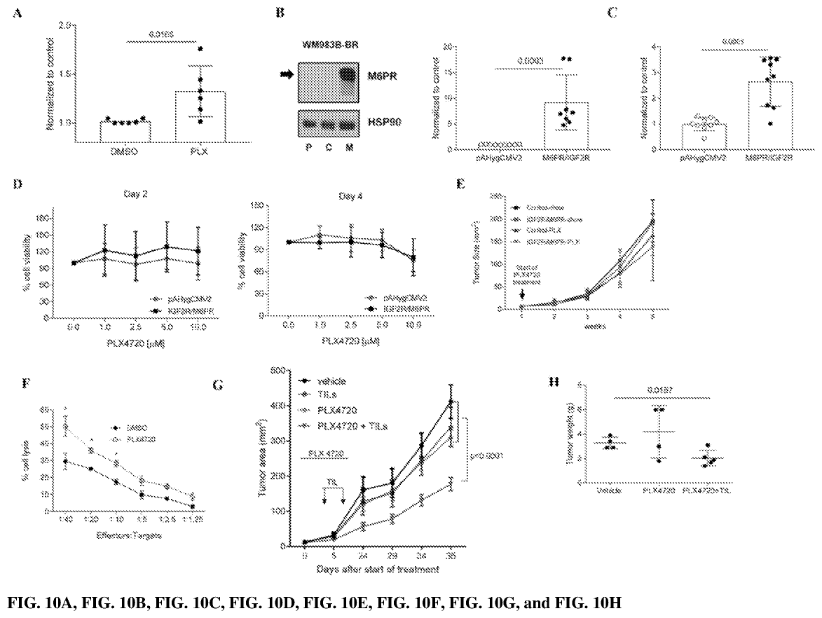

We asked whether PLX4720 treatment could increase the uptake of GrzB by BRAFi resistant cells. WM35-BR cells were treated with DMSO and 10 μM PLX4720 overnight, and after confirming upregulation of M6PR on the cell surface, pretreated cells were incubated with inactive GrzB at 37° C. for one hour and intracellular GrzB levels assessed by flow cytometry. PLX4720 treated cells had significantly higher amount of GrzB than DMSO treated cells (FIG. 10A). Overexpression of M6PR in WM983B-BR cells (WM983B-BR-M6PR/IGF2R) (FIG. 10B) markedly increased GrzB uptake (FIG. 10C). Over-expression of M6PR did not improve the survival of WM983B-BR cells in response to treatment with PLX4720 in vitro (FIG. 10D). WM983B-BR control and M6PR-overexpressing cells were subcutaneously injected into opposite flanks of immune-deficient NSG mice, and when tumors became palpable, mice were treated with PLX4720. No difference in tumor growth was seen (FIG. 10E) supporting the conclusion that M6PR overexpression by itself does not reverse resistance of melanoma cells to BRAF inhibition.

We next tested whether PLX-induced M6PR up-regulation on the cell surface of resistant cells sensitized them to HLA-matched TIL. Overnight treatment of WM35-BR with PLX4720 increased sensitivity of tumor cells to TIL in cytotoxicity assay (FIG. 10F). We asked whether BRAFi could sensitize resistant tumors to TILs in vivo. WM35-BR cells were injected subcutaneously to the flanks of immune deficient NOD/SCID mice and when tumors became palpable, mice were split to 4 groups. Mice treated with vehicle alone, mice treated with 50 mg/kg PLX4720 for 10 days, mice treated with CD8+ T-cell enriched TIL i.v. twice at a 4 day interval and mice treated with combination of PLX4720 and TILs. TIL injections alone or PLX4720 did not affect tumor growth. However, when PLX4720 treatment was combined with TIL transfer, tumor growth was significantly decreased (FIG. 10G) and the tumor weight of this group was significantly lower than the vehicle-treated or PLX-treated groups (FIG. 10H), indicating that PLX4720—potentiated the anti-tumor effect of TIL in vivo in resistant tumor cells.

(5) The Effect of BRAF Inhibitor Vemurafenib on M6PR Expression in Melanoma Patients

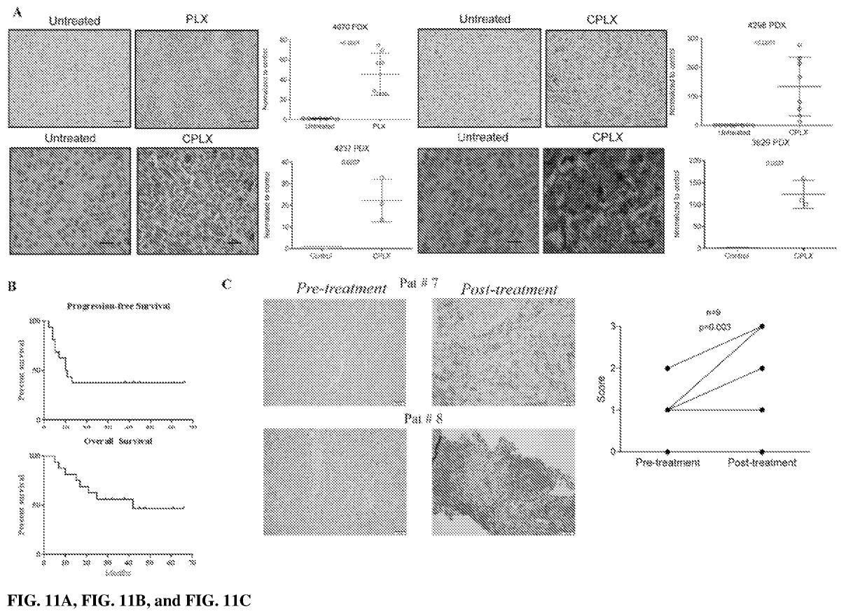

To test the effect of therapy on M6PR expression in clinical samples, we established patient derived xenografts (PDX) from four patients who were either treatment naïve (4237, 3929), or progressed on BRAFi (4070) or combination of BRAFi and MEKi (4298). Mice with PDX were left untreated or treated for 3 weeks with single agent BRAFi (PLX-PLX4720) or combination with MEKi (CPLXPLX4720 and PD0325901). In all four cases, treatment with inhibitors caused marked up-regulation of M6PR in tumors (FIG. 11A).

A clinical trial of vemurafenib and TIL has been performed in patients with metastatic melanoma. Previously completed clinical trial of ACT with TIL was associated with a 26% objective response rate based upon intention to treat. However, it had 32% patient attrition rate largely due to disease progression prior to ACT. Therefore, the goals of combining vemurafenib with TIL ACT were to reduce patient attrition and to improve clinical responses. Clinical trial (NCT01659151) was conducted at H. Lee Moffitt Cancer Center in 2014-2017. Seventeen subjects with BRAF V600-mutated tumors were accrued with subject characteristics and TIL phenotype listed in Table 1. All subjects started vemurafenib the day after tumor harvest, and one subject dropped out prior to ACT due to inadequate TIL growth, representing an attrition rate of 6%. The null hypothesis for this endpoint was 32% (based upon the historical track record at H. Lee Moffitt Cancer Center). Thus, this trial demonstrated a marked improvement in the attrition rate of patients after tumor harvest who were due for TIL therapy.

TABLE 1
Clinical characteristics of patients enrolled to the study

| | | | | | | Number | Response | Response | Duration of |
|---|---|---|---|---|---|---|---|---|---|
| Patient | | | | Previous | Site of | of TIL | at 12 | at 12 | Vem treatment |
| No. | Age | Gender | Stage | Therapy | Resection | infused | Weeks | Months | (months) |
| 1 | 18 | F | M1c | αCTLA-4, | Soft tissue | 2.0E+10 | PD | PD | 6 |
| | | | | αPD1, IL2 | | | | | |
| 2 | 31 | F | M1c | none | Soft tissue | 9.1E+09 | PD | PD | 3 |
| 3 | 50 | F | M1c | none | Soft tissue | 8.1E+10 | PR | PR | 18 |
| 4 | 38 | M | M1c | none | Soft tissue | 4.3E+10 | PR | PD | 8 |
| 5 | 68 | F | M1c | none | Inguinal node | 5.2E+10 | PR | PR | 1 |
| 6 | 55 | M | M1c | None | Axillary node | 8.6E+10 | PR | PR | 12 |
| 7 | 68 | F | M1c | none | Soft tissue and | 3.1E+10 | PR | PD | 3 |
| | | | | | axillary node | | | | |
| 8 | 42 | M | M1c | none | Soft tissue | 3.9E+10 | PR | PD | 9 |
| 9 | 47 | M | IIIC | none | Axillary node | 5.0E+10 | CR | CR | 24 |
| 10 | 41 | M | IIIC | none | Axillary node | 5.2E+10 | SD | PD | 13 |
| 11 | 49 | M | M1c | αCTLA-4, | axillary node | 5.3E+10 | PR | PR | 20 |
| | | | | αPD1 | | | | | |
| 12 | 47 | F | M1c | none | Neck node | 6.5E+10 | PD | PD | 4 |
| 13 | 53 | M | M1c | adjuvant | Axillary node | 3.1E+10 | PR | PR | 18 |
| | | | | αCTLA-4 & | | | | | |
| | | | | αPD1 | | | | | |
| 14 | 53 | M | IIIC | none | Soft tissue | 5.6E+10 | SD | PD | 9 |
| 15 | 39 | F | M1b | none | Inguinal node | 7.3E+10 | PR | PD | 7 |
| 16 | 35 | M | M1c | none | Axillary node | 1.1E+11 | PD | PD | 1 |

Vemurafenib was held during the ACT regimen and was resumed upon clinical recovery for up to 2 years. There were no treatment-related deaths. Toxicity was expected and included the typical vemurafenib and adoptive cell therapy-related toxicities of bone marrow suppression, neutropenic fever, chronic fatigue neuropathy, and skin toxicity (Table 2). There was no long-term toxicity that was definitely related to therapy aside from the above. Six of the 16 accrued patients (38%) manifested an objective response at the pre-specified endpoint of 12 months. The null hypothesis for this endpoint was <30% (based upon historical track record). The median progression-free survival was 10.5 months, and the median overall survival was 42 months (FIG. 11B). The 6 objective responders achieved an overall survival that ranged from 38 to 66 months. One responding subject who developed symptomatic disease not meeting criteria for progression underwent surgical resection and is without evidence of disease after 48 months of follow up without additional treatment. One subject who achieved a complete response died from a cause independent from melanoma after 42 months.

TABLE 2
Grade 3+ Adverse Events in treated patients

| Adverse Event¹ | Grade 3 | Grade 4 |
|---|---|---|
| Neutropenia | 0 | 16 |
| Lymphopenia | 0 | 16 |
| Thrombocytopenia | 2 | 12 |
| Febrile neutropenia | 11 | 0 |
| Rash | 7 | 0 |
| Anemia | 6 | 0 |
| Vascular catheter-related thrombosis | 4 | 2 |
| Cutaneous squamous cell carcinoma | 5 | 0 |
| Pulmonary edema | 4 | 0 |
| Hyponatremia | 3 | 0 |
| Emesis | 3 | 0 |
| Hypertension | 3 | 0 |
| Primary cutaneous melanoma | 1 | 0 |
| Cutaneous basal cell carcinoma | 1 | 0 |
| Oliguria | 1 | 0 |
| Hypotension | 1 | 0 |
| Diarrhea | 1 | 0 |
| Anasarca | 1 | 0 |
| Confusion | 1 | 0 |
| Transaminitis | 1 | 0 |
| Vasovagal reaction | 1 | 0 |
| Hyperbilirubinemia | 1 | 0 |

¹Note
that one patient was treated with vemurafenib but not with ACT

To assess the effect of the treatment on the expression of M6PR in tumors from patients on this trial we evaluated pre- and on-treatment biopsies. Biopsies from nine patients treated with vemurafenib followed by ACT were available for evaluation. On-treatment biopsies were obtained from 10-321 days after initiation of vemurafenib treatment (median 77 days). Expression of M6PR was noted in both tumor cells and lymphocytes (FIG. 11C). M6PR tumor scores increased in 8 samples from 7 patients and were stable in 2 samples from 2 patients (FIG. 11C) while on treatment (p=0.003). There were no significant changes in either CD4 or CD8 lymphocytes within or surrounding the tumor after initiation of vemurafenib treatment. Thus, BRAF inhibitor up-regulated M6PR in tumors from cancer patients and in combination with BRAF inhibition and ACT demonstrated promising clinical results.

c) Discussion

In this study, we demonstrated that BRAFi sensitized human melanoma cells to killing by CTLs. Because of widespread resistance to BRAFi, new combination modalities are necessary, and one possible approach would be to combine BRAFi with ACT. We tested the hypothesis that BRAFi can cause up-regulation of M6PR and thus sensitize tumor cells to TILs. It is shown herein that the combination of vemurafenib with ACT showed an enhanced antitumor effect in immunocompetent mice bearing BRAF^{V600E} mutant SM1 melanoma cells. Vemurafenib treatment did not cause any increase in the expansion or tumor infiltration of adoptively transferred T cells. However, the antitumor activity of antigen-specific T cells was significantly increased after vemurafenib treatment, indicating that understanding the mechanism behind the improvement in the anti-tumor effect of combination therapy can lead to more effective therapy for metastatic melanoma. Herein is demonstrated, in models of lung cancer and lymphoma, that several chemotherapeutic drugs (paclitaxel, doxorubicin, cisplatin) as well as radiation therapy can potentiate the anti-tumor effect of immunotherapy via up-regulation of M6PR on the tumor cell surface. Since M6PR can bind GrzB, this may explain enhanced tumor cell killing in a perforin-independent manner. In the current study, we tested the effect of BRAFi (PLX4720) on induction of M6PR in different human melanoma cell lines. PLX4720 and vemurafenib has been shown to promote the anti-tumor effects of T cells. PLX4720 treatment decreased tumor CCL2 expression in BRAF^{V600E} mouse melanoma transplants. PLX4720 did not directly increase tumor immunogenicity, but caused a robust increase in the CD8+T/FoxP3+CD4+ T cell ratio and in NK cells. Using TILs isolated from cancer patients and HLA matched melanoma cell lines with overexpressed or deleted M6PR, we determined that upregulation of M6PR was directly responsible for BRAFi-induced increased sensitivity of tumor cells to CTLs. M6PR up-regulation was mediated by autophagy induced by various stress signals. It is possible that a similar mechanism was involved in the impact of BRAFi. In the current work we did not study the specific mechanism of M6PR upregulation, but focused on the impact of this effect on the treatment of BRAFi resistant cells. We observed that BRAFi caused upregulation of M6PR in both sensitive and resistant melanoma cells. The magnitude of up-regulation was similar in vitro, whereas the BRAFi effect in sensitive cells was stronger. This is consistent with a higher basal level of the receptor being expressed in resistant cells. Although cell surface M6PR returned to the pre-treatment level in sensitive cells after 24 hours of treatment, in resistant cells MPR levels were still significantly higher in comparison to DMSO treated cells after 24 hours. Considering that BRAFi resistance is induced by exposing cells to sustained and increased doses of inhibitor, continuous stress may result in higher levels of the receptor. Consistent with stress-induced upregulation of the receptor were also the results of stronger up-regulation of M6PR by BRAFi in hypoxia. Expression of M6PR did not affect the sensitivity of melanoma cells to BRAFi since neither upregulation nor deletion of the receptor affected the viability of cells exposed to BRAFi. However, augmentation of M6PR levels was a major mechanism regulating increased sensitivity of tumor cells to CTLs, since deletion of the receptor abrogated killing of tumor cells by TIL.

Several clinical trials have demonstrated the clinical efficacy of the BRAFi and MEKi combination in metastatic melanoma patients. Furthermore, dabrafenib and trametinib combination therapy was shown to increase the expression of melanoma antigen, T cell cytotoxicity markers and CD8+ T cell infiltration in biopsies from metastatic melanoma patients in comparison to vemurafenib treatment alone, indicating that the combination of BRAFi and MEKi can augment the antitumor effect of immunotherapy. In our study, we observed that although trametinib alone did not induce M6PR in sensitive cell lines, in combination with PLX4720, up-regulation of M6PR was stronger than PLX4720 alone. In contrast, combining trametinib with PLX4720 did not enhance M6PR up-regulation in BRAFi resistant cells. These results further show that the effect of BRAFi on M6PR expression is not associated with canonical signaling via MAPK.

The results of a clinical trial demonstrated that treatment with BRAFi caused marked up-regulation of M6PR in tumors. Our data in PDX showed that up-regulation of M6PR can be observed in tumors after failure of BRAFi or with the combination of BRAFi and MEKi therapy indicating that in these patients addition of ACT can be beneficial. This study demonstrated that targeting of BRAF in BRAF^{V600E} mutant melanoma sensitizes tumor cells to killing by CTLs at least in part via up-regulation of M6PR. Combining ACT with BRAFi treatment in patients who progressed on BRAFi and MEKi therapy is therapeutically useful.

### D. REFERENCES

Ascierto P A, Kirkwood J M, Grob J J, Simeone E, Grimaldi A M, Maio M, et al. The role of BRAF V600 mutation in melanoma. J Transl Med. 2012; 10:85.
Baruch E N, Berg A L, Besser M J, Schachter J, Markel G. Adoptive T cell therapy: An overview of obstacles and opportunities. Cancer. 2017; 123:2154-62.
Boni A, Cogdill A P, Dang P, Udayakumar D, Njauw C N, Sloss C M, et al. Selective BRAFV600E inhibition enhances T-cell recognition of melanoma without affecting lymphocyte function. Cancer Res. 2010; 70:5213-9.
Chapman P B, Hauschild A, Robert C, Haanen J B, Ascierto P, Larkin J, et al. Improved survival with vemurafenib in melanoma with BRAF V600E mutation. N Engl J Med. 2011; 364:2507-16.
Comin-Anduix B, Chodon T, Sazegar H, Matsunaga D, Mock S, Jalil J, et al. The oncogenic BRAF kinase inhibitor PLX4032/RG7204 does not affect the viability or function of human lymphocytes across a wide range of concentrations. Clin Cancer Res. 2010; 16:6040-8.
Deniger D C, Kwong M L, Pasetto A, Dudley M E, Wunderlich J R, Langhan M M, et al. A Pilot Trial of the Combination of Vemurafenib with Adoptive Cell Therapy in Patients with Metastatic Melanoma. Clin Cancer Res. 2017; 23:351-62.
Eroglu Z, Ribas A. Combination therapy with BRAF and MEK inhibitors for melanoma: latest evidence and place in therapy. Therapeutic advances in medical oncology. 2016; 8:48-56.
Flaherty K T, Infante J R, Daud A, Gonzalez R, Kefford R F, Sosman J, et al. Combined BRAF and MEK inhibition in melanoma with BRAF V600 mutations. N Engl J Med. 2012; 367:1694-703.
Frederick D T, Piris A, Cogdill A P, Cooper Z A, Lezcano C, Ferrone C R, et al. BRAF inhibition is associated with enhanced melanoma antigen expression and a more favorable tumor microenvironment in patients with metastatic melanoma. Clin Cancer Res. 2013; 19:1225-31.
Hall M, Liu H, Malafa M, Centeno B, Hodul P J, Pimiento J, et al. Expansion of tumor infiltrating lymphocytes (TIL) from human pancreatic tumors. J Immunother Cancer. 2016; 4:61.
Hartsough E, Shao Y, Aplin A E. Resistance to RAF inhibitors revisited. J Invest Dermatol. 2014; 134:319-25.
Kim A, Cohen M S. The discovery of vemurafenib for the treatment of BRAF-mutated metastatic melanoma. Expert opinion on drug discovery. 2016; 11:907-16.
Kim S, Ramakrishnan R, Lavilla-Alonso S, Chinnaiyan P, Rao N, Fowler E, et al. Radiation induced autophagy potentiates immunotherapy of cancer via up-regulation of mannose 6-phosphate receptor on tumor cells in mice. Cancer Immunol Immunother. 2014; 63:1009-21.
Knight D A, Ngiow S F, Li M, Parmenter T, Mok S, Cass A, et al. Host immunity contributes to the anti-melanoma activity of BRAF inhibitors. J Clin Invest. 2013; 123:1371-81.
Koya R C, Mok S, Otte N, Blacketor K J, Comin-Anduix B, Tumeh P C, et al. BRAF inhibitor vemurafenib improves the antitumor activity of adoptive cell immunotherapy. Cancer Res. 2012; 72:3928-37.
Krepler C, Sproesser K, Brafford P, Beqiri M, Garman B, Xiao M, et al. A Comprehensive Patient-Derived Xenograft Collection Representing the Heterogeneity of Melanoma. Cell reports. 2017; 21:1953-67.
Long G V, Flaherty K T, Stroyakovskiy D, Gogas H, Levchenko E, de Braud F, et al. Dabrafenib plus trametinib versus dabrafenib monotherapy in patients with metastatic BRAF V600E/K-mutant melanoma: long-term survival and safety analysis of a phase 3 study. Ann Oncol. 2017; 28:1631-9.
Long G V, Grob J J, Nathan P, Ribas A, Robert C, Schadendorf D, et al. Factors predictive of response, disease progression, and overall survival after dabrafenib and trametinib combination treatment: a pooled analysis of individual patient data from randomised trials. The lancet oncology. 2016; 17:1743-54.
Merhavi-Shoham E, Itzhaki O, Markel G, Schachter J, Besser M J. Adoptive Cell Therapy for Metastatic Melanoma. Cancer journal (Sudbury, Mass. 2017; 23:48-53.
Motyka B, Korbutt G, Pinkoski M J, Heibein J A, Caputo A, Hobman M, et al. Mannose 6-phosphate/insulin-like growth factor II receptor is a death receptor for granzyme B during cytotoxic T cell-induced apoptosis. Cell. 2000; 103:491-500.
Nazarian R, Shi H, Wang Q, Kong X, Koya R C, Lee H, et al. Melanomas acquire resistance to B-RAF (V600E) inhibition by RTK or N-RAS upregulation. Nature. 2010; 468:973-7.
Paraiso K H, Xiang Y, Rebecca V W, Abel E V, Chen Y A, Munko A C, et al. PTEN loss confers BRAF inhibitor resistance to melanoma cells through the suppression of BIM expression. Cancer Res. 2011; 71:2750-60.
Pilon-Thomas S, Kuhn L, Ellwanger S, Janssen W, Royster E, Marzban S, et al. Efficacy of adoptive cell transfer of tumor-infiltrating lymphocytes after lymphopenia induction for metastatic melanoma. J Immunother. 2012; 35:615-20.
Probst O C, Karayel E, Schida N, Nimmerfall E, Hehenberger E, Puxbaum V, et al. The mannose 6-phosphate-binding sites of M6P/IGF2R determine its capacity to suppress matrix invasion by squamous cell carcinoma cells. Biochem J. 2013; 451:91-9.
Ramakrishnan R, Assudani D, Nagaraj S, Hunter T, Cho H I, Antonia S, et al. Chemotherapy enhances tumor cell susceptibility to CTL-mediated killing during cancer immunotherapy in mice. J Clin Invest. 2010; 120:1111-24.
Ramakrishnan R, Huang C, Cho H I, Lloyd M, Johnson J, Ren X, et al. Autophagy induced by conventional chemotherapy mediates tumor cell sensitivity to immunotherapy. Cancer Res. 2012; 72:5483-93.
Schadendorf D, Long G V, Stroiakovski D, Karaszewska B, Hauschild A, Levchenko E, et al. Three-year pooled analysis of factors associated with clinical outcomes across dabrafenib and trametinib combination therapy phase 3 randomised trials. Eur J Cancer. 2017; 82:45-55.
Shannan B, Watters A, Chen Q, Mollin S, Dorr M, Meggers E, et al. PIM kinases as therapeutic targets against advanced melanoma. Oncotarget. 2016; 7:54897-912.
Sosman J A, Kim K B, Schuchter L, Gonzalez R, Pavlick A C, Weber J S, et al. Survival in BRAF V600-mutant advanced melanoma treated with vemurafenib. N Engl J Med. 2012; 366:707-14.
Wan P T, Garnett M J, Roe S M, Lee S, Niculescu-Duvaz D, Good V M, et al. Mechanism of activation of the RAF-ERK signaling pathway by oncogenic mutations of B-RAF. Cell. 2004; 116:855-67.
Wilmott J S, Long G V, Howle J R, Haydu L E, Sharma R N, Thompson J F, et al. Selective BRAF inhibitors induce marked T-cell infiltration into human metastatic melanoma. Clin Cancer Res. 2012; 18:1386-94.
Yee C. Adoptive T cell therapy: points to consider. Curr Opin Immunol. 2018; 51:197-203.

1. A method of treating a cancer in a subject using an adoptive T cell therapy, the method comprising administering to the subject a BRAF inhibitor and an adoptive T cell therapy, wherein administration of the BRAF inhibitor increases insulin-like growth factor II receptor (IGF2R).

2. A method of increasing the sensitivity of a cancer in a subject to adoptive T cell therapy, the method comprising administering to the subject receiving adoptive T cell therapy a BRAF inhibitor, wherein administration of the BRAF inhibitor increases insulin-like growth factor II receptor (IGF2R).

3. The method of claim 1, wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer melanoma, colorectal carcinoma, papillary thyroid carcinoma, hairy cell leukemia, and Langerhans cell histiocytosis, pleomorphic xanthoastrocytoma, ganglioglioma, epithelioid glioblastoma, and gliomas diagnosed at a younger age; melanoma, colorectal, thyroid, and Non-small cell lung cancer (NSCLC), as well as hairy cell leukemia.

4. The method of claim 1, wherein the cancer is a BRAF inhibitor resistant cancer.

5. The method of claim 1, wherein the adoptively transferred T cells are chimeric antigen receptor T cells (CAR T cells) or tumor infiltrating lymphocytes (TlLs).

6. The method of claim 1, wherein the BRAF inhibitor comprises sorafenib, vemurafenib, dabrafenib, and/or encorafenib.

7. A method of treating a cancer in a subject with an adoptive T cell therapy, the method comprising administering to the subject an lGF2R agonist and an adoptive T cell therapy.

8. A method of increasing the sensitivity of a cancer in a subject to adoptive T cell therapy, the method comprising administering to the subject an lGF2R agonist.

9. The method of claim 7, wherein the lGF2R agonist comprises clenbuterol.

10. The method of claim 7, wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer melanoma, colorectal carcinoma, papillary thyroid carcinoma, hairy cell leukemia, and Langerhans cell histiocytosis, pleomorphic xanthoastrocytoma, ganglioglioma, epithelioid glioblastoma, and gliomas diagnosed at a younger age; melanoma, colorectal, thyroid, and Non-small cell lung cancer (NSCLC), as well as hairy cell leukemia.

11. The method of claim 7, wherein the cancer is a BRAF inhibitor resistant cancer.

12. The method of claim 7, wherein the adoptively transferred T cells are chimeric antigen receptor T cells (CAR T cells) or tumor infiltrating lymphocytes (TlLs).

13. The method of claim 7 further comprising administering to the subject a BRAF inhibitor.

14. The method of claim 1, wherein the cancer is a BRAF inhibitor resistant cancer and wherein the method further comprises administering to the subject a MEK inhibitor.

15. The method of claim 2, wherein the cancer is a BRAF inhibitor resistant cancer.

16. The method of claim 2, wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer melanoma, colorectal carcinoma, papillary thyroid carcinoma, hairy cell leukemia, and Langerhans cell histiocytosis, pleomorphic xanthoastrocytoma, ganglioglioma, epithelioid glioblastoma, and gliomas diagnosed at a younger age; melanoma, colorectal, thyroid, and Non-small cell lung cancer (NSCLC), as well as hairy cell leukemia.

17. The method of claim 2, wherein the BRAF inhibitor comprises sorafenib, vemurafenib, dabrafenib, and/or encorafenib.

18. The method of claim 8, wherein the cancer is a BRAF inhibitor resistant cancer.

19. The method of claim 8, wherein the cancer is selected from the group of cancers consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer melanoma, colorectal carcinoma, papillary thyroid carcinoma, hairy cell leukemia, and Langerhans cell histiocytosis, pleomorphic xanthoastrocytoma, ganglioglioma, epithelioid glioblastoma, and gliomas diagnosed at a younger age; melanoma, colorectal, thyroid, and Non-small cell lung cancer (NSCLC), as well as hairy cell leukemia.

20. The method of claim 8, wherein the lGF2R agonist comprises clenbuterol.


SEQUENCE LISTING (1 sequence)

SEQ ID NO: 1 - DNA, 20 bp, Artificial Sequence
  Synthetic construct
agtccgggcccggcgcgatg